(19) United States
(12) Patent Application Publication
(10) Pub. No.: US 2026/0225064 A1
(43) Pub. Date: Aug. 6, 2026

(54) COMPOUND AIR LOOP REACTOR

(71) Applicant: ARIZONA BOARD OF REGENTS ON BEHALF OF THE UNIVERSITY OF ARIZONA, Tucson, AZ (US)

(72) Inventors: Joel L. Cuello, Tucson, AZ (US); Michael Mason, Tucson, AZ (US)

(21) Appl. No.: 19/154,762

(22) PCT Filed: Feb. 9, 2024
(86) PCT No.: PCT/US2024/015238

§ 371 Date: Aug. 7, 2025

Related U.S. Application Data
(60) Provisional application No. 63/484,304, filed on Feb. 10, 2023.

(51) Int. Cl. B01J 19/24

(52) U.S. Cl. CPC B01J 19/246; B01J 19/2435

(57) ABSTRACT

A compound air loop reactor for circulating and mixing a substance. The reactor may comprise a chamber. The chamber may comprise one or more inlets, a plurality of riser-downcomer pairs. Each riser-downcomer pair may comprise a riser channel configured to transport the substance upwards, a downcomer channel configured to transport the substance downwards, and one or more connecting channels. Each connecting channel may fluidly couple a riser channel of the one or more riser channels to a downcomer channel of the one or more downcomer channels. The reactor may further comprise one or more outlets fluidly coupled to the chamber.

### CROSS-REFERENCES TO RELATED APPLICATIONS

This application claims benefit of U.S. Provisional Application No. 63/484,304 filed Feb. 10, 2023, the specification of which is incorporated herein in its entirety by reference.

### FIELD OF THE INVENTION

The present invention is directed to compound bioreactors for circulating bulk liquid and gas for photoautotrophic, mixotrophic or heterotrophic growth and production of microalgae and other microorganisms (bacteria, fungi, etc.) as well as cell cultures of plants, animals, insects, and others.

### BACKGROUND OF THE INVENTION

The Compound Air Loop Reactors (CALR) were conceived as reactors that could be used for the photoautotrophic, mixotrophic or heterotrophic growth and production of microalgae and other microorganisms (bacteria, fungi, etc.) as well as cell cultures of plants, animals, insects and others.

The photoautotrophic growth of microorganisms or cells is enabled by the photosynthetic capacity of the chlorophyll-containing microorganisms or cells, whereby carbon dioxide (CO₂), through photosynthetic carbon fixation, serves as the carbon (or food) source. Photoautotrophic growth requires light-driven photosynthesis to occur. A steady supply of supplemental CO₂, in addition to what is naturally present in air, when light is available also promotes culture growth.

By contrast, heterotrophic growth takes place when the microorganisms or cells, in the absence of photosynthetic CO₂ fixation, rely on exogenous carbon-based molecules, typically sugars such as glucose or sucrose, present in the liquid culture medium as their carbon (or food) source. Heterotrophic growth necessitates a sterile or axenic growth environment to avoid culture contamination; otherwise, unwanted and competing bacteria and other microorganisms would grow in the culture owing to the presence of the carbon-based food source. This mode of growth also requires a steady supply of oxygen (O₂) which the microorganisms or cells need as they break down the carbon-based molecules through the process of respiration. Since light is not essential, heterotrophic production is generally carried out in darkness. Mixotrophic growth takes place when the microorganisms or cells grow both photoautotrophically and mixotrophically.

The Compound Air Loop Reactor (CALR) was designed to have the following advantages: (i) Low-shear culture environment; (ii) Superior liquid mixing; (iii) Efficient gas mass transfer; (iv) Transparent walls for photoautotrophic/mixotrophic production; and (v) Transparent or non-transparent walls and sterile environment for heterotrophic production.

### BRIEF SUMMARY OF THE INVENTION

It is an objective of the present invention to provide devices that allow for the circulation and mixing of both bulk liquid and gas for the growth and production of microorganisms and cell cultures, as specified in the independent claims. Embodiments of the invention are given in the dependent claims. Embodiments of the present invention can be freely combined with each other if they are not mutually exclusive.

The Compound Air Loop Reactors (CALR) are original pneumatic airlift reactors designed specifically for the photoautotrophic, mixotrophic, or heterotrophic growth and production of microalgae and other microorganisms (bacteria, fungi, etc.) as well as cell cultures of plants, animals, insects, and others. The CALR's were designed to have the advantages of providing a low-shear culture environment, superior liquid mixing, and efficient gas mass transfer, among others. The CALR's, with their unique characteristics, encompass: (1) the 360 Series Internal Air Loop Reactor (360-SIAR); (2) the 360 Series External Air Loop Reactor (360-SEAR); (3) the Parallel Internal Air Loop Reactor (PIAR); and (4) the Parallel External Air Loop Reactor (PEAR).

The three types of conventional air loop reactors, wherein bulk liquid flow and recirculation are caused by sparged gas, typically air, through the riser (R) and downcomer (D), are shown in FIGS. 1A-1C. While the Internal Air Loop Concentric Tube Reactor was the basis for the patented Accordion Air Loop (or H-Accordion) Reactor, the present invention is based on the Internal Air Loop Split Reactor and the External Air Loop Reactor.

The present invention features a compound air loop reactor for circulating bulk liquid and a gas. The reactor may comprise a chamber. The chamber may comprise one or more inlets, a plurality of riser-downcomer pairs. Each riser-downcomer pair may comprise a riser channel configured to transport the bulk liquid and gas upwards, a downcomer channel configured to transport the bulk liquid and gas downwards, and one or more connecting channels. Each connecting channel may fluidly couple a riser channel of the one or more riser channels to a downcomer channel of the one or more downcomer channels. The reactor may further comprise one or more outlets fluidly coupled to the chamber.

In some embodiments, at least one of the plurality of riser-downcomer pairs may further comprise an internal divider separating the riser channel from the downcomer channel. These embodiments are directed to internal air loop reactors. In other embodiments, at least one of the plurality of riser-downcomer pairs further comprise a dividing space separating the riser channel from the downcomer channel. These embodiments are directed to external air loop reactors. In some embodiments, the chamber is cyclical overall. In other embodiments, the chamber comprises a plurality of parallel cycles each connected by a connecting channel of the one or more connecting channels.

One of the unique and inventive technical features of the present invention is the implementation of multiple linked riser-downcomer pairs in a bulk-liquid and gas-circulating bioreactor system. Without wishing to limit the invention to any theory or mechanism, it is believed that the technical feature of the present invention advantageously provides for a low-shear culture environment, superior liquid mixing, and efficient gas mass transfer. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A-1C show a plurality of types of conventional air loop reactors wherein bulk liquid flow and recirculation are caused by sparged gas, typically air, through the riser and downcomer.

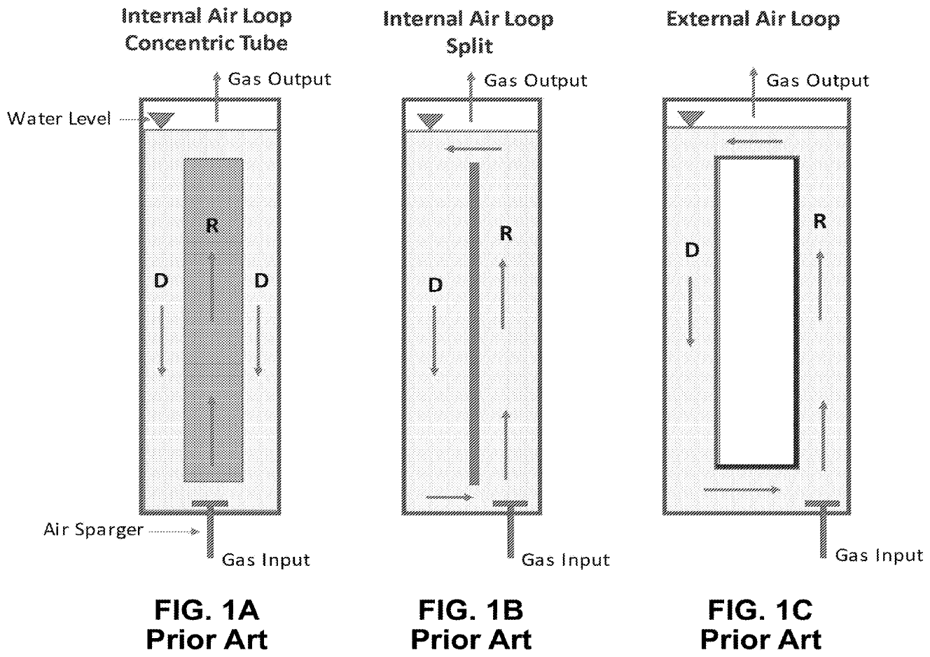

FIG. 2 shows an embodiment of the 360 Series Internal Air Loop Reactor (360-SIAR) with two pairs (2′) of risers and downcomers.

FIG. 3 shows an embodiment of the 360 Series Internal Air Loop Reactor (360-SIAR) with three pairs (3′) of risers and downcomers.

FIG. 4 shows a three-dimensional illustration of an embodiment of the 360 Series Internal Air Loop Reactor (360-SIAR) with two pairs (2′) of risers and downcomers.

FIG. 5 shows an embodiment of the 360 Series External Air Loop Reactor (360-SEAR) with two pairs (2′) of risers and downcomers.

FIG. 6 shows an embodiment of the 360 Series External Air Loop Reactor (360-SEAR) with three pairs (3′) of risers and downcomers.

FIG. 7 shows an embodiment of the Parallel Internal Air Loop Reactor (PIAR) with two pairs (2′) of risers and downcomers.

FIG. 8 shows an embodiment of the Parallel Internal Air Loop Reactor (PIAR) with three pairs (3′) of risers and downcomers.

FIG. 9 shows an embodiment of the Parallel External Air Loop Reactor (PEAR) with two pairs (2′) of risers and downcomers.

FIG. 10 shows an embodiment of the Parallel External Air Loop Reactor (PEAR) with three pairs (3°) of risers and downcomers.

FIGS. 11A-11C show geometric shapes for the flow scaffold or guide, which may be straight, sinusoidal (FIG. 11A), trapezoidal (FIG. 11B), zigzag (FIG. 11C), etc.

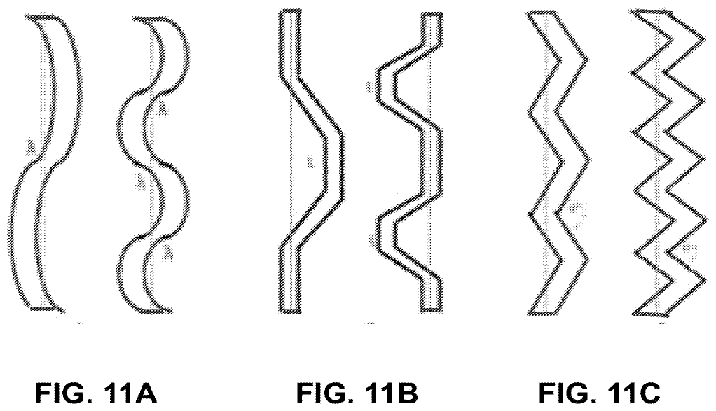

FIG. 12 shows a diagram of the compound air loop reactor of the present invention showing an example of the dimensions and materials used.

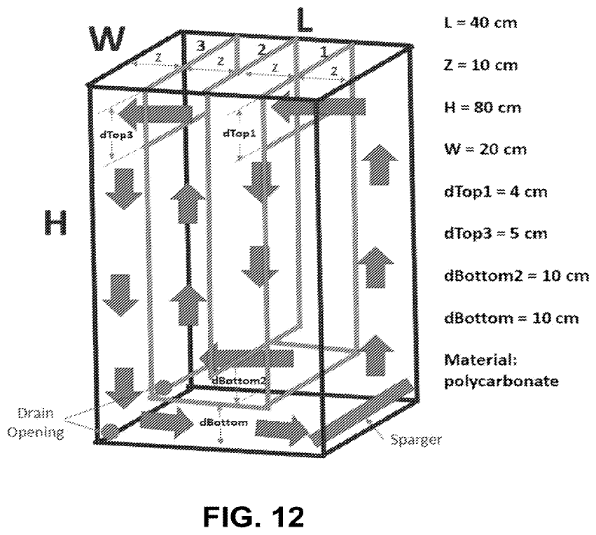

FIG. 13 shows a photo of a 5.1-Liter laboratory-scale prototype of the Compound Air Loop Reactor (CALR) of the present invention.

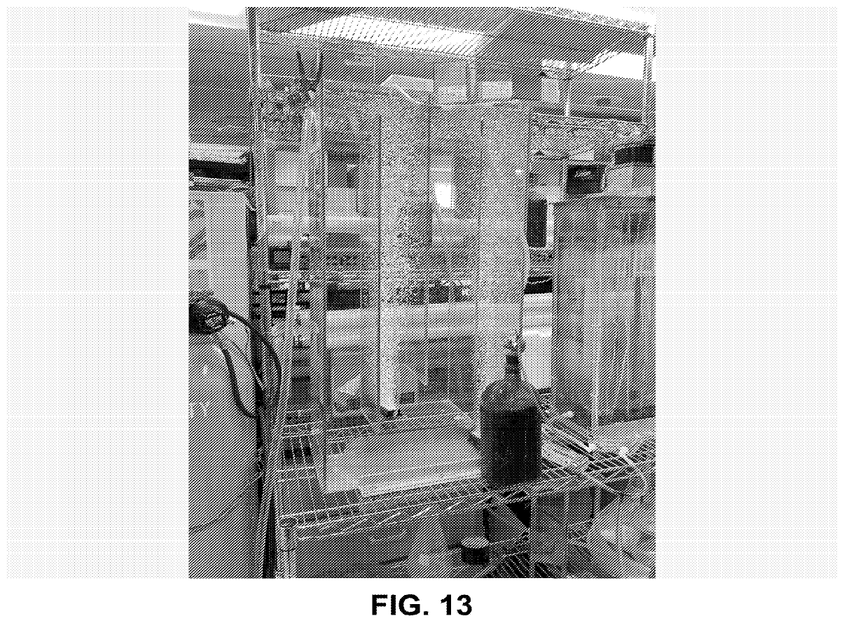

### DETAILED DESCRIPTION OF THE INVENTION

Following is a list of elements corresponding to a particular element referred to herein:
100 reactor
110 chamber
112 inlet
113 riser channel
114 downcomer channel
115 connecting channel
120 outlet

The term “air sparger” is defined herein as a device configured to inject air or oxygen through a contaminated aquifer.

The present invention features a compound air loop reactor (100) for circulating and mixing a substance. In some embodiments, the reactor (100) may comprise a chamber (110). In some embodiments, the chamber (110) may comprise one or more riser channels (113) configured to transport the substance upwards, one or more downcomer channels (114) configured to transport the substance downwards, and one or more connecting channels (115). Each connecting channel (115) may be configured to fluidly couple at least one riser channel (113) to at least one downcomer channel (114). The chamber (110) may further comprise one or more inlets (112) fluidly coupled to the one or more riser channels (113), the one or more downcomer channels (114), the one or more connecting channels (115), or a combination thereof. The chamber (110) may further comprise one or more outlets (120) fluidly coupled to the one or more riser channels (113), the one or more downcomer channels (114), the one or more connecting channels (115), or a combination thereof.

In some embodiments, the reactor (100) may further comprise a dividing wall configured to separate at least one riser channel of the one or more riser channels (113) from at least one downcomer channel of the one or more downcomer channels (114). In some embodiments, the reactor (100) may further comprise a dividing space configured to separate at least one riser channel of the one or more riser channels (113) from at least one downcomer channel of the one or more downcomer channels (114). In some embodiments, the chamber (110) may be cyclical.

In some embodiments, the chamber (110) may comprise a plurality of parallel cycles each comprising a riser channel of the one or more riser channels (113) and a downcomer channel of the one or more downcomer channels (114) connected by at least one connecting channel of the one or more connecting channels (115). In some embodiments, at least one inlet of the one or more inlets (112) may comprise an air sparger. In some embodiments, one or more walls of the chamber (110) may comprise flow guides. In some embodiments, a shape of the flow guides may comprise a straight shape, a sinusoidal shape, a trapezoidal shape, a zigzag shape, or a combination thereof. In some embodiments, a material of the flow guides may comprise metal, glass, polyvinyl chloride, polycarbonate, polymer, or a combination thereof. In some embodiments, a material of the chamber (110) may comprise metal, glass, polyvinyl chloride, polycarbonate, polymer, or a combination thereof. In some embodiments, the reactor (100) may further comprise one or more lighting components, wherein the one or more lighting components are disposed within the chamber (110), outside of the chamber (110), or a combination thereof.

The present invention features a compound air loop reactor (100) for circulating and mixing a substance. In some embodiments, the reactor (100) may comprise a chamber (110). In some embodiments, the chamber (110) may comprise one or more riser-downcomer pairs. Each riser-downcomer pair may comprise an inlet (112), a riser channel (113) fluidly coupled to the inlet, configured to transport the substance upwards, a downcomer channel (114), configured to transport the substance downwards, and two connecting channels (115). Each connecting channel (115) may be configured to fluidly couple the riser channel (113) to the downcomer channel (114) such that a cycle is formed. Each riser-downcomer pair may further comprise an internal divider component configured to separate the riser channel (113) from the downcomer channel (114). Each riser-downcomer pair may be configured to be at least partially separated from other riser-downcomer pairs by an external dividing component. The chamber may further comprise one or more outlets (120) fluidly coupled to each riser-downcomer pair.

In some embodiments, each internal dividing component, each external dividing component, or a combination thereof may comprise a dividing wall. In some embodiments, each internal dividing component, each external dividing component, or a combination thereof may comprise a dividing space. In some embodiments, each inlet (112) of the one or more inlets (112) may comprise an air sparger. In some embodiments, the reactor (100) may further comprise one or more lighting components, wherein the one or more lighting components are disposed within the chamber (110), outside of the chamber (110), or a combination thereof.

The present invention features a reactor (100) for transporting a substance, the reactor (100) comprising a plurality of alternating riser channels (113) fluidly coupled to a plurality of downcomer channels (114), arranged such that the riser channels (113) are alternating with the downcomer channels (114). The riser channels (113) may be configured to transport the substance in one direction and the downcomer channels (114) may be configured to transport the substance in another direction. In some embodiments, the reactor (100) may further comprise a dividing wall configured to separate at least one riser channel of the plurality of riser channels (113) from at least one downcomer channel of the plurality of downcomer channels (114). In some embodiments, the reactor (100) may further comprise a dividing space configured to separate at least one riser channel of the plurality of riser channels (113) from at least one downcomer channel of the plurality of downcomer channels (114). In some embodiments, the reactor (100) may further comprise a chamber (110) configured to contain the plurality of alternating riser channels (113) and the plurality of downcomer channels (114).

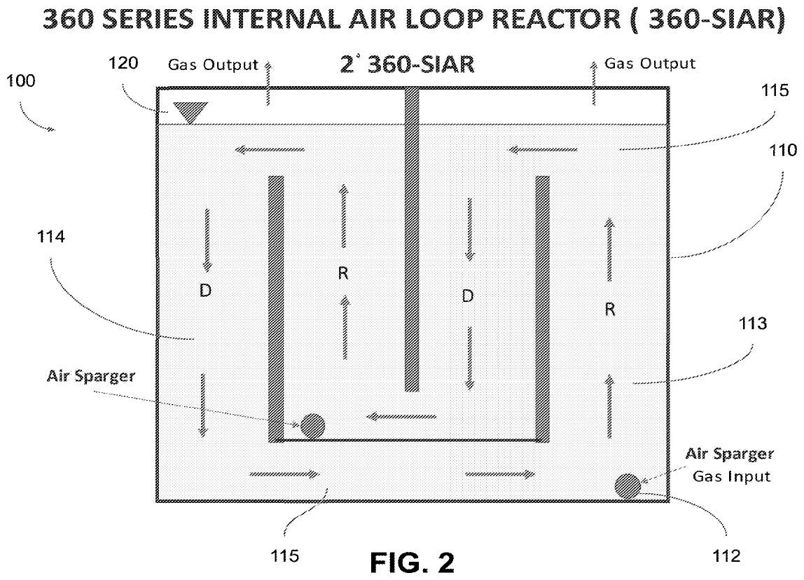

Referring now to FIGS. 2-10, the present invention features a compound air loop reactor (100) for circulating and mixing a substance. In some embodiments, the substance comprises a bulk liquid, a gas, a culture of biological cells or a combination thereof. The reactor (100) may comprise a chamber (110). The chamber (110) may comprise one or more inlets (112), a plurality of riser-downcomer pairs. Each riser-downcomer pair may comprise a riser channel (113) configured to transport the substance upwards, a downcomer channel (114) configured to transport the substance downwards, and one or more connecting channels (115). Each connecting channel (115) may fluidly couple a riser channel (113) of the one or more riser channels (113) to a downcomer channel (114) of the one or more downcomer channels (114). The reactor (100) may further comprise one or more outlets (120) fluidly coupled to the chamber (110).

In some embodiments, at least one of the plurality of riser-downcomer pairs may further comprise an internal divider separating the riser channel (113) from the downcomer channel (114). These embodiments are directed to internal air loop reactors. In other embodiments, at least one of the plurality of riser-downcomer pairs further comprise a dividing space separating the riser channel (113) from the downcomer channel (114). These embodiments are directed to external air loop reactors.

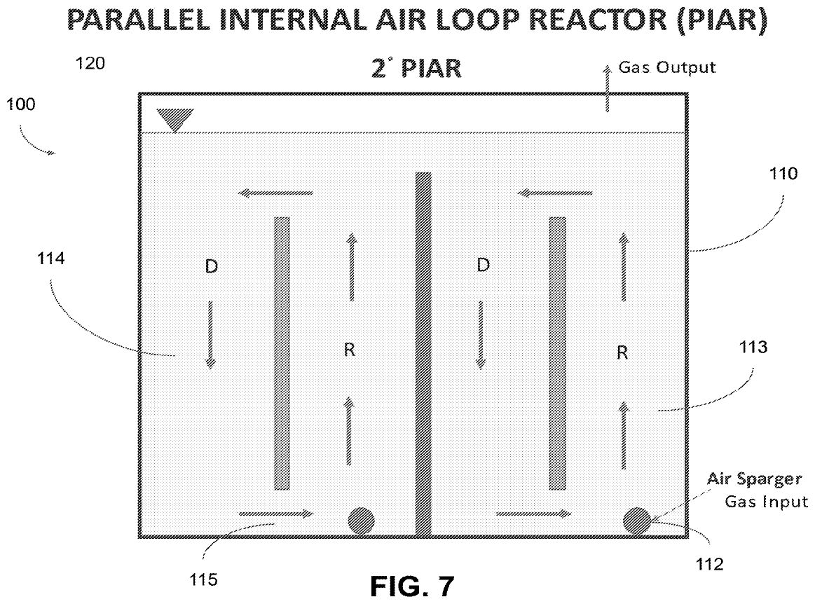

In some embodiments, the chamber (110) is cyclical overall (FIGS. 2-6). In other embodiments, the chamber (110) comprises a plurality of parallel cycles each connected by a connecting channel (115) of the one or more connecting channels (115) (FIGS. 7-10).

Referring now to FIGS. 2-6, the present invention features a compound air loop reactor (100) for circulating and mixing a substance. In some embodiments, the substance comprises a bulk liquid, a gas, or a combination thereof. The reactor (100) may comprise a chamber (110) comprising one or more inlets (112) and a plurality of riser-downcomer pairs. Each riser-downcomer pair may comprise a riser channel (113) configured to transport the substance upwards, a downcomer channel (114) configured to transport the substance downwards, and one or more connecting channels (115). Each connecting channel (115) may fluidly couple a riser channel (113) of the one or more riser channels (113) to a downcomer channel (114) of the one or more downcomer channels (114). The chamber (110) may be cyclical. The reactor may further comprise one or more outlets (120) fluidly coupled to the chamber (110).

Referring now to FIGS. 7-10, the present invention features a compound air loop reactor (100) for circulating and mixing a substance. In some embodiments, the substance comprises a bulk liquid, a gas, or a combination thereof. The reactor (100) comprising a chamber (110) comprising a plurality of riser-downcomer pairs. Each riser-downcomer pair may comprise an inlet (112), a riser channel (113), configured to transport the substance upwards, a downcomer channel (114), configured to transport the substance downwards, and two connecting channels (115). Each connecting channel (115) may fluidly couple a riser channel (113) of the one or more riser channels (113) to a downcomer channel (114) of the one or more downcomer channels (114).

Each riser-downcomer pair is at least partially separated from the others by a dividing component. The chamber (110) may further comprise one or more outlets (120) fluidly coupled to each riser-downcomer pair.

Each inlet (112) of the one or more inlets (112) may comprise an air sparger. One or more walls of the chamber (110) may comprise flow guides. A shape of the flow guides may comprise a straight shape, a sinusoidal shape, a trapezoidal shape, or a zigzag shape. In some embodiments, a material of the chamber (110) and/or the flow guides may comprise metal, glass, polyvinyl chloride, polycarbonate, polymer, or any other rigid material. The reactor (100) may further comprise one or more lighting components, wherein the one or more lighting components are disposed within the chamber (110), outside of the chamber (110), or a combination thereof.

The present invention features a compound air loop reactor (100) for circulating and mixing a substance, the reactor (100) comprising a plurality of riser channels (113) interchangeably coupled to a plurality of downcomer channels (114). The plurality of riser channels (113) may transport the substance in a direction that is different from that of the plurality of downcomer channels (114).

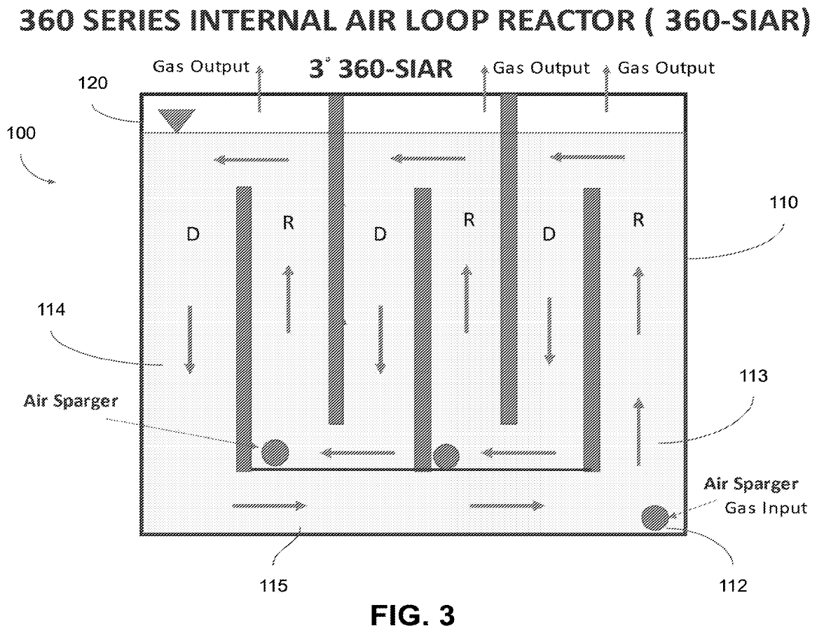

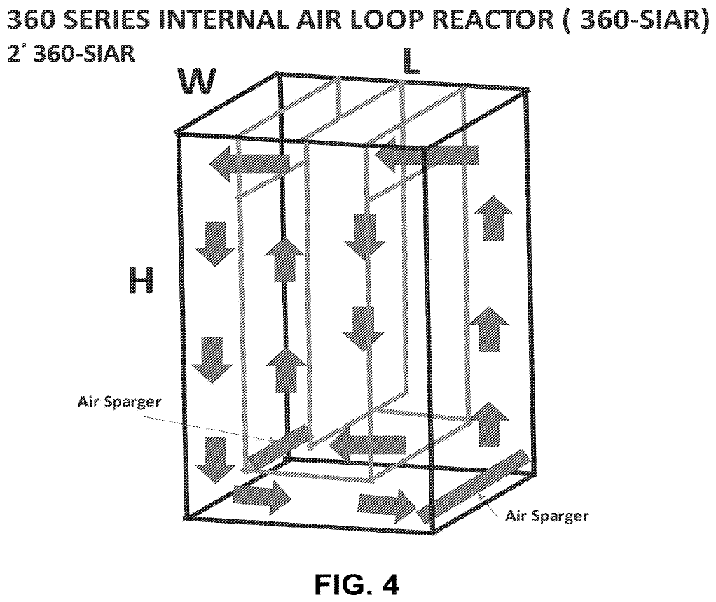

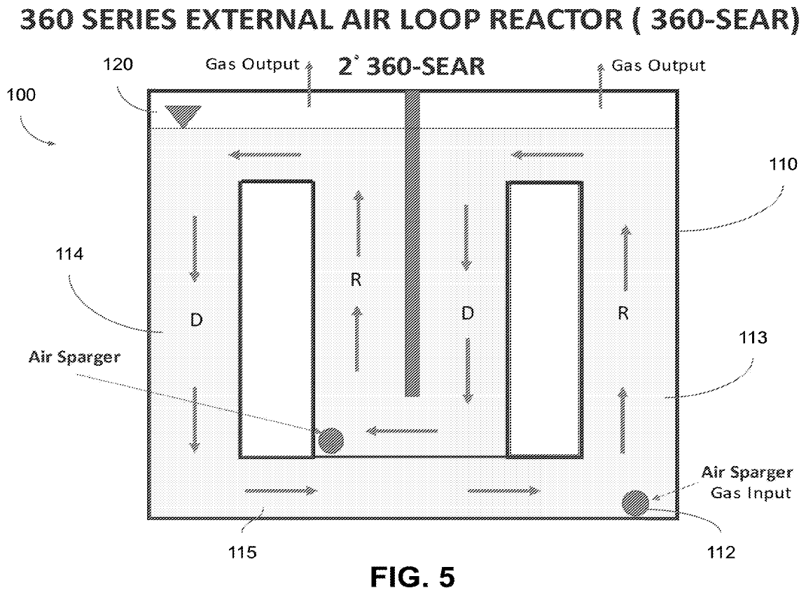

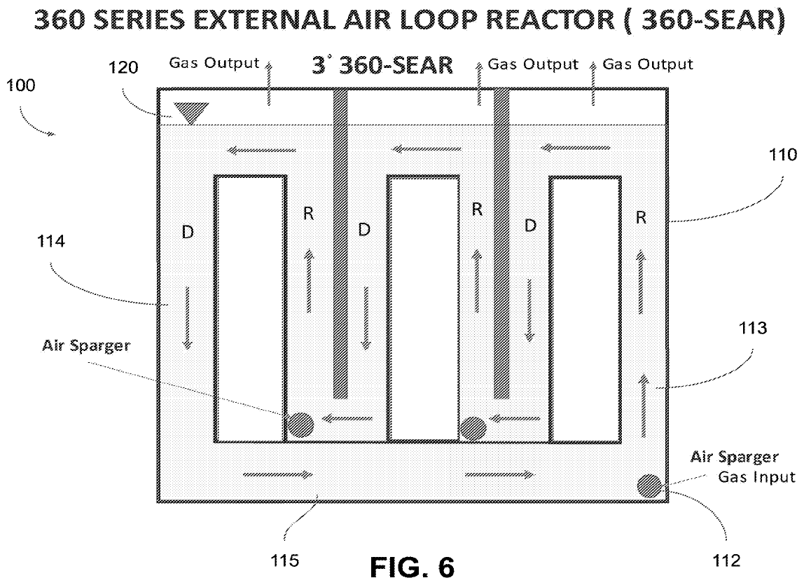

FIGS. 2-3 show embodiments of the 360 Series Internal Air Loop Reactor (360-SIAR) with two (2′) and three (3′) pairs, respectively, of riser and downcomer channels. The 360-SIAR may have as many pairs of R and D as practicable. A three-dimensional illustration of the 360 Series Internal Air Loop Reactor (360-SIAR) with two pairs (2′) of riser and downcomer channels is shown in FIG. 4. FIGS. 5-6 show embodiments of the 360 Series External Air Loop Reactor (360-SEAR) with two (2′) and three (3′) pairs, respectively, of riser and downcomer channels. The 360-SEAR may have as many pairs of risers and downcomers as practicable.

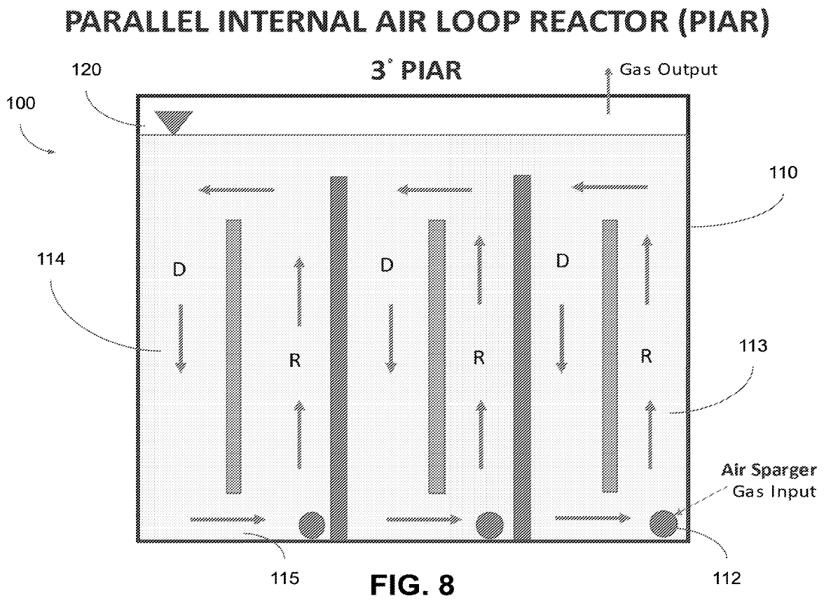

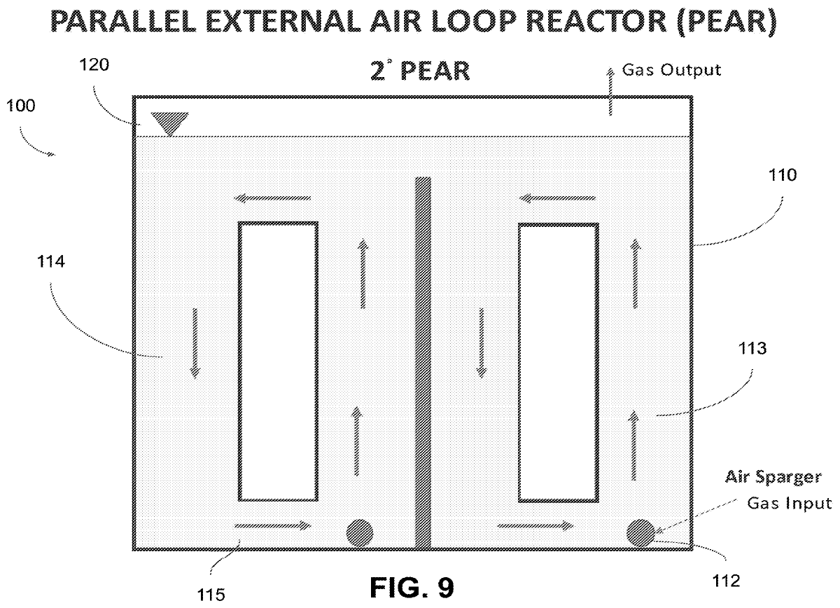

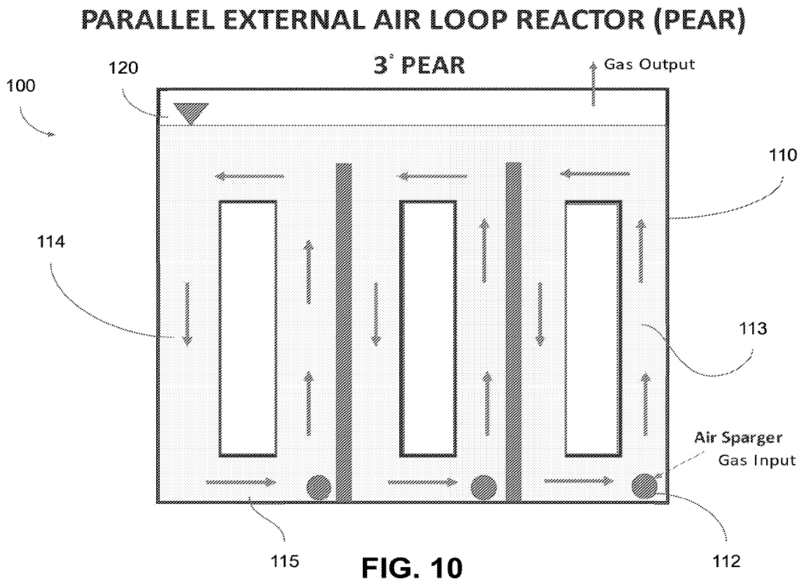

FIGS. 7-8 show embodiments of the Parallel Internal Air Loop Reactor (PIAR) with two (2′) and three (3′) pairs, respectively, of riser and downcomer. The PIAR may have as many pairs of risers and downcomers as practicable. FIGS. 9-10 show embodiments of the Parallel External Air Loop Reactor (PEAR) with two (2′) and three (3′) pairs, respectively, of riser and downcomer channels. The PEAR may have as many pairs of risers and downcomers as practicable.

In some embodiments, the reactor of the present invention may range in scale from 1 L capacity to 1000 L capacity or greater. A geometric configuration of the reactor may be that of a panel or a cylinder, with a cross-sectional area of a rectangle, square, circle, ellipse, etc. The chamber may comprise fixed or removable air-loop flow scaffolds or guides. These flow guides may be straight, sinusoidal, trapezoidal, zigzag, etc. A degree, or number of pairs, of risers and downcomers may range from two to as many as practicable.

A material of the reactor and flow guides may be rigid (e.g., metal, glass, PVC, polycarbonate, polymer, etc.) or soft (LDPE, etc.), and may be transparent, translucent, or opaque. The one or more air spargers may be of any material (ceramic, polymer, etc.) and any geometric shape (cylindrical, circular, etc.). The lighting components of the reactor may be internal or external to the reactor vessel and may be of any type (LED, etc.). The reactor (100) of the present invention may have a height of about 2 meters, a length of about 1 meter, and a width of about 0.5 meters.

In some embodiments, each riser channel may comprise a lower portion, a middle portion, and an upper portion. In some embodiments, the lower portion of the riser channel may comprise a lower third of a height of the riser channel. In some embodiments, the middle portion of the riser channel may comprise a middle third of the height of the riser channel. In some embodiments, the upper portion of the riser channel may comprise an upper third of the height of the riser channel. In some embodiments, the riser channel may only comprise a lower portion and an upper portion. In some embodiments, the lower portion may comprise a lower half of the height of the riser channel. In some embodiments, the upper portion may comprise an upper half of the height of the riser channel.

In some embodiments, each downcomer channel may comprise a lower portion, a middle portion, and an upper portion. In some embodiments, the lower portion of the downcomer channel may comprise a lower third of a height of the downcomer channel. In some embodiments, the middle portion of the downcomer channel may comprise a middle third of the height of the downcomer channel. In some embodiments, the upper portion of the downcomer channel may comprise an upper third of the height of the downcomer channel. In some embodiments, the downcomer channel may only comprise a lower portion and an upper portion. In some embodiments, the lower portion may comprise a lower half of the height of the downcomer channel. In some embodiments, the upper portion may comprise an upper half of the height of the downcomer channel.

In some embodiments, the one or more inlets (112) may be disposed at the lower portion of a riser channel, the middle portion of the riser channel, the upper portion of the riser channel, or a combination thereof. In some embodiments, the one or more inlets (112) may be disposed at the lower portion of a downcomer channel, the middle portion of the downcomer channel, the upper portion of the downcomer channel, or a combination thereof. In some embodiments, the one or more outlets (120) may be disposed at the lower portion of a riser channel, the middle portion of the riser channel, the upper portion of the riser channel, or a combination thereof. In some embodiments, the one or more outlets (120) may be disposed at the lower portion of a downcomer channel, the middle portion of the downcomer channel, the upper portion of the downcomer channel, or a combination thereof.

In some embodiments, the one or more connecting channels (115) may connect the lower portion of the riser channel to the lower portion of the downcomer channel, the middle portion of the downcomer channel, the upper portion of the downcomer channel, or a combination thereof. In some embodiments, the one or more connecting channels (115) may connect the middle portion of the riser channel to the lower portion of the downcomer channel, the middle portion of the downcomer channel, the upper portion of the downcomer channel, or a combination thereof. In some embodiments, the one or more connecting channels (115) may connect the upper portion of the riser channel to the lower portion of the downcomer channel, the middle portion of the downcomer channel, the upper portion of the downcomer channel, or a combination thereof.

In some embodiments, each riser channel may be coupled to one or more downcomer channels, one or more riser channels, one or more connecting channels, or a combination thereof. In some embodiments, each downcomer channel may be coupled to one or more downcomer channels, one or more riser channels, one or more connecting channels, or a combination thereof. In some embodiments, each connecting channel may be coupled to one or more downcomer channels, one or more riser channels, one or more connecting channels, or a combination thereof.

In some embodiments, the compound air loop reactor of the present invention may comprise a three-dimensional chamber containing the various channels and dividers. In some embodiments, the chamber may comprise a length of 30 to 50 cm. In some embodiments, the chamber may comprise a width of 10 to 30 cm. In some embodiments, the chamber may comprise a height of 60 to 100 cm. In some embodiments, the one or more riser channels may comprise a diameter of 5 to 15 cm. In some embodiments, the one or more downcomer channels may comprise a diameter of 5 to 15 cm. In some embodiments, the one or more inlets may comprise a diameter of 3 to 12 cm. In some embodiments, the one or more outlets may comprise a diameter of 3 to 12 cm. In some embodiments, the one or more connecting channels may comprise a diameter of 3 to 12 cm. In some embodiments, the lower connecting channels may have a larger diameter than the higher connecting channels.

In some embodiments, a shape of the chamber may comprise a rectangular prism, a triangular prism, a sphere, or any three-dimensional shape suitable for containing the one or more riser channels, the one or more downcomer channels, and the one or more connecting channels. In some embodiments, a cross-section of the one or more riser channels may comprise a rectangle, a circle, a triangle, or any polygonal shape. In some embodiments, a cross-section of the one or more downcomer channels may comprise a rectangle, a circle, a triangle, or any polygonal shape. In some embodiments, a cross-section of the one or more connecting channels may comprise a rectangle, a circle, a triangle, or any polygonal shape.

In some embodiments, the substance may comprise a gas and a liquid. In some embodiments, the gas may comprise carbon dioxide (CO₂). In some embodiments, the liquid may comprise a mixture of nutrients. In some embodiments, the substance may comprise a gas, a liquid, a powder, an aerosol, a plasma, a flowable solid, or a combination thereof.

In some embodiments, the reactor of the present invention may further comprise one or more pumps fluidly coupled to at least one of the one or more inlets, at least one of the one or more outlets, or a combination thereof, configured to move the substance through the channels of the reactor. In some embodiments, the reactor of the present invention may further comprise one or more filters disposed within the one or more riser channels, the one or more downcomer channels, the one or more connecting channels, or a combination thereof for affecting the substance as it passes through said filters. In some embodiments, the reactor may further comprise one or more reservoirs fluidly coupled to at least one of the one or more inlets, configured to contain at least a portion of the substance. In some embodiments, the reactor may further comprise one or more reservoirs fluidly coupled to at least one of the one or more outlets, configured to contain the substance after mixing within the reactor.

In some embodiments, a material of the chamber (110) may comprise metal, glass, polyvinyl chloride, polycarbonate, polymer, composites, or a combination thereof.

In some embodiments, a material of the one or more riser channels may comprise metal, glass, polyvinyl chloride, polycarbonate, polymer, composites, or a combination thereof. In some embodiments, a material of the one or more downcomer channels may comprise metal, glass, polyvinyl chloride, polycarbonate, polymer, composites, or a combination thereof. In some embodiments, a material of the one or more connecting channels may comprise metal, glass, polyvinyl chloride, polycarbonate, polymer, composites, or a combination thereof.

In some embodiments, at least one of the one or more riser channels may comprise an interior coating comprising cellulose derivatives. In some embodiments, at least one of the one or more downcomer channels may comprise an interior coating comprising cellulose derivatives. In some embodiments, at least one of the one or more connecting channels may comprise an interior coating comprising cellulose derivatives.

In some embodiments, at least one side of the container may be at least partially transparent. In some embodiments, at least one of the one or more riser channels may comprise at least one side that is at least partially transparent. In some embodiments, at least one of the one or more downcomer channels may comprise at least one side that is at least partially transparent. In some embodiments, at least one of the one or more connecting channels may comprise at least one side that is at least partially transparent. In some embodiments, one or more lighting fixtures may be disposed such that light is able to enter the reactor through one or more at least partially transparent sides. In some embodiments, the term “at least partially transparent” may refer to any surface capable of allowing light to pass through (e.g. glass, polysulfone, polycarbonate).

### EXAMPLE

The following is a non-limiting example of the present invention. It is to be understood that said example is not intended to limit the present invention in any way. Equivalents or substitutes are within the scope of the present invention.

The mixing times for the compound air loop reactor (CALR) of the present invention and a standard control bubble reactor (CBR) were compared in an experimental trial. The results (three replications per treatment) are shown in the table below:

| | Compound Air Loop | Control Bubble |
|---|---|---|
| | Reactor | Reactor |
| Low Air Flow Rate | 4.0 ± 0.3 min | 0.5 ± 0.1 min |
| High Air Flow Rate | 2.4 ± 0.1 min | 0.4 ± 0.0 min |

The results of the mixing-time experiments showed that the average mixing time for the CALR significantly exceeded that for the CBR either for the low air flow rate treatment or the high air flow rate treatment of air sparging. This is explained by the CALR possessing rigid up-and-down, hence, defined liquid flow channels made up of two pairs of riser and downcomer; thus, making it longer for the liquid medium to achieve a well-mixed condition.

Contrastively, the CBR lacked the foregoing rigid up-and-down, hence, defined liquid flow channels, enabling its liquid medium to achieve a significantly quicker well-mixed condition. The mixing-time results underscored the CALR's critical advantage of a well-defined and stable liquid flow pattern which, more importantly, is preserved on scale up to any larger volume. By contrast, the CBR's liquid flow pattern is known to become both unstable and variable on scale-up, and is expected to result in significantly longer mixing time, that is, if a well-mixed condition could ever be achieved, at very large volumes.

Although there has been shown and described the preferred embodiment of the present invention, it will be readily apparent to those skilled in the art that modifications may be made thereto which do not exceed the scope of the appended claims.

Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A compound air loop reactor (100) for circulating and mixing a substance, the reactor (100) comprising:
a. a chamber (110) comprising:
i. one or more riser channels (113) configured to transport the substance upwards;
ii. one or more downcomer channels (114) configured to transport the substance downwards;
iii. one or more connecting channels (115), wherein each connecting channel (115) is configured to fluidly couple at least one riser channel (113) to at least one downcomer channel (114); and
iv. one or more inlets (112) fluidly coupled to the one or more riser channels (113), the one or more downcomer channels (114), the one or more connecting channels (115), or a combination thereof;
v. one or more outlets (120) fluidly coupled to the one or more riser channels (113), the one or more downcomer channels (114), the one or more connecting channels (115), or a combination thereof.

2. The reactor (100) of claim 1 further comprising a dividing wall configured to separate at least one riser channel of the one or more riser channels (113) from at least one downcomer channel of the one or more downcomer channels (114).

3. The reactor (100) of claim 1 further comprising a dividing space configured to separate at least one riser channel of the one or more riser channels (113) from at least one downcomer channel of the one or more downcomer channels (114).

4. The reactor (100) of claim 1, wherein the chamber (110) is cyclical.

5. The reactor (100) of claim 1, wherein the chamber (110) comprises a plurality of parallel cycles each comprising a riser channel of the one or more riser channels (113) and a downcomer channel of the one or more downcomer channels (114) connected by at least one connecting channel of the one or more connecting channels (115).

6. The reactor (100) of claim 1, wherein at least one inlet of the one or more inlets (112) comprises an air sparger.

7. The reactor (100) of claim 1, wherein one or more walls of the chamber (110) comprise flow guides.

8. The reactor (100) of claim 7, wherein a shape of the flow guides comprises a straight shape, a sinusoidal shape, a trapezoidal shape, a zigzag shape, or a combination thereof.

9. The reactor (100) of claim 7, wherein a material of the flow guides comprises metal, glass, polyvinyl chloride, polycarbonate, polymer, or a combination thereof.

10. The reactor (100) of claim 1, wherein a material of the chamber (110) comprises metal, glass, polyvinyl chloride, polycarbonate, polymer, or a combination thereof.

11. The reactor (100) of claim 1 further comprising one or more lighting components, wherein the one or more lighting components are disposed within the chamber (110), outside of the chamber (110), or a combination thereof.

12. A compound air loop reactor (100) for circulating and mixing a substance, the reactor (100) comprising:
a. a chamber (110) comprising:
i. one or more riser-downcomer pairs, each riser-downcomer pair comprising:
A. an inlet (112);
B. a riser channel (113) fluidly coupled to the inlet (112), configured to transport the substance upwards;
C. a downcomer channel (114), configured to transport the substance downwards;
D. two connecting channels (115), wherein each connecting channel (115) is configured to fluidly couple the riser channel (113) to the downcomer channel (114) such that a cycle is formed; and
E. an internal divider component configured to separate the riser channel (113) from the downcomer channel (114);
wherein each riser-downcomer pair is configured to be at least partially separated from other riser-downcomer pairs by an external dividing component; and
ii. one or more outlets (120) fluidly coupled to each riser-downcomer pair.

13. The reactor (100) of claim 12, wherein each internal dividing component, each external dividing component, or a combination thereof comprises a dividing wall.

14. The reactor (100) of claim 12, wherein each internal dividing component, each external dividing component, or a combination thereof comprises a dividing space.

15. The reactor (100) of claim 12, wherein each inlet (112) of the one or more inlets (112) comprises an air sparger.

16. The reactor (100) of claim 12 further comprising one or more lighting components, wherein the one or more lighting components are disposed within the chamber (110), outside of the chamber (110), or a combination thereof.

17. A reactor (100) for transporting a substance, the reactor (100) comprising a plurality of alternating riser channels (113) fluidly coupled to a plurality of downcomer channels (114), arranged such that the riser channels (113) are alternating with the downcomer channels (114), wherein the riser channels (113) are configured to transport the substance in one direction and the downcomer channels (114) are configured to transport the substance in another direction.

18. The reactor (100) of claim 17 further comprising a dividing wall configured to separate at least one riser channel of the plurality of riser channels (113) from at least one downcomer channel of the plurality of downcomer channels (114).

19. The reactor (100) of claim 17 further comprising a dividing space configured to separate at least one riser channel of the plurality of riser channels (113) from at least one downcomer channel of the plurality of downcomer channels (114).

20. The reactor (100) of claim 17 further comprising a chamber (110) configured to contain the plurality of alternating riser channels (113) and the plurality of downcomer channels (114).
